(19) United States
(12) Patent Application Publication
(10) Pub. No.: US 2015/0119371 A1
(43) Pub. Date: Apr. 30, 2015

(54) TOPICAL PHARMACEUTICAL COMPOSITIONS OF ANTIBIOTICS AND STEROIDAL ANTI INFLAMMATORY AGENTS

(71) Applicant: Micro Labs Limited, Bangalore (IN)

(72) Inventors: Rajesh KSHIRSAGAR, Bangalore (IN); Sachin MUNDADE, Bangalore (IN)

(21) Appl. No.: 14/528,124

(22) Filed: Oct. 30, 2014

(30) Foreign Application Priority Data
Oct. 30, 2013 (IN) 4897/CHE/2013

(51) Int. Cl. A61K 9/00; A61K 31/573; A61K 47/12; A61K 47/18; A61K 47/02; A61K 47/10; A61K 31/496; A61K 47/38

(52) U.S. Cl. CPC A61K 9/0046; A61K 31/496; A61K 31/573; A61K 47/38; A61K 47/186; A61K 47/02; A61K 47/10; A61K 47/12
USPC 514/171

(57) ABSTRACT

The present invention relates to sterile, storage-stable topically administrable otic pharmaceutical compositions comprising one or more antibiotic(s) and one or more steroidal anti-inflammatory agent(s) and a process for preparation thereof. The topical pharmaceutical composition of the present invention further comprises ionic polymer and pharmaceutically acceptable excipients thereof. The topical pharmaceutical composition of invention is physically stable and can be easily re-suspended.

### FIELD OF THE INVENTION

The present invention relates to pharmaceutical compositions. In particular, this invention relates to sterile, storage-stable topically administrable otic pharmaceutical compositions comprising one or more antibiotic(s) and one or more steroidal anti-inflammatory agent(s) and a process for preparation thereof.

### BACKGROUND OF THE INVENTION

U.S. Pat. Nos. 6,284,804 and 6,359,016 discloses topically administrable sterile ophthalmic and otic suspensions of Ciprofloxacin and Dexamethasone, marketed by Alcon under tradename CiproDex® for treatment of acute otitis media and acute otitis externa, containing nonionic polymer, a nonionic surfactant and an ionic tonicity agent. The nonionic polymer incorporated in these suspensions is hydroxyethyl cellulose in an amount of 0.2% weight (wt.)

U.S. Pat. Nos. 5,843,930 and 5,965,549 discloses non-ototoxic, non-irritating and non-sensitizing aqueous otic suspension for the treatment of acute otitis externa and otitis media, particularly otorrhea. The composition comprises nonionic polymer, a nonionic surfactant and a nonionic tonicity agent. The '549 patent teaches that “aqueous solutions of ionic polymers such as carbopol and sodium carboxymethylcellulose were found to have undesirable viscosity variability with ionic level and pH” (see Col. 5, lines 17-21). For compatibility with ciprofloxacin hydrochloride solubility, such agents were preferably non-ionic and unaffected by pH and ionic level (see Col. 5, lines 14-16). The composition comprises ciprofloxacin in an amount effective for antibacterial action; a non-ionic viscosity augmenter unaffected by pH and ionic level in an amount effective for augmenting viscosity of the composition to a viscosity greater than that of water; and water sufficient to produce a liquid composition. The viscosity augmenter is chosen from the group consisting of methylcellulose, polyvinyl alcohol, and glycerine (see Col. 2, lines 52-56).

U.S. Pat. No. 6,066,292 provides method for sterilizing a pharmaceutical suspension of a water-insoluble pharmaceutical like steroids including Hydrocortisone and Dexamethasone by preparation of three pre-mixes comprising i) a first sterile pre-mix of heat-sterilized aqueous solution of a viscosity enhancer (b) a second pre-mix having sterile-filtered aqueous solution of a mixture of a pharmaceutically-active compound (c) third sterile pre-mix containing heat-sterilized mixture of water, a water-insoluble pharmaceutical, and at least a partial amount of an sodium chloride to provide a sub-saturated solution, and adding under aseptic conditions an aqueous surfactant (d) combining all three pre-mixes in sterile fashion to achieve a sterile suspended pharmaceutical formulation like sterile suspension of Ciprofloxacin and Hydrocortisone. The viscosity enhancers used in this composition, such as polyvinyl alcohol or lecithin is non-ionic in nature.

While there are compositions available, still there remains a need to develop sterile, storage-stable topically administrable otic pharmaceutical composition comprising antiinflammatory agent(s) and antibiotic agent(s) having excellent physical stability and are characterized by their easy and ready resuspendability.

The present inventors have now found that sterile storage-stable topically administrable otic pharmaceutical compositions comprising one or more antibiotic(s), one or more steroidal anti-inflammatory agent(s) and one or more ionic polymer(s) provides excellent physical stability and are characterized by their easy and ready resuspendability.

### SUMMARY OF THE INVENTION

The present invention is directed to a sterile, storage-stable topically administrable otic pharmaceutical compositions comprising one or more antibiotic(s) and one or more steroidal anti-inflammatory agent(s).

In one aspect, the present invention provides sterile, storage-stable topically administrable otic pharmaceutical compositions comprising:

(a) one or more antibiotic(s),
(b) one or more steroidal anti-inflammatory agent(s),
(c) one or more Ionic polymer(s).

In yet another aspect, the present invention provides a pharmaceutical composition comprising fixed dose combination comprising one or more antibiotic(s) and one or more steroidal anti-inflammatory agent(s).

In yet another aspect, the present invention provides a pharmaceutical composition comprising fixed dose combination comprising one or more antibiotic(s), one or more steroidal anti-inflammatory agent(s) and one or more Ionic polymer(s).

In yet another aspect, the present invention provides a stable topically administrable otic pharmaceutical compositions comprising:

(a) one or more antibiotic(s);
(b) one or more steroidal anti-inflammatory agent(s);
(c) one or more ionic polymer(s), wherein the composition has an osmolality in the range from 270 to 330 mOsml/kg.

In yet another aspect, the present invention provides a stable topically administrable otic pharmaceutical compositions comprising:

(a) one or more antibiotic(s);
(b) one or more steroidal anti-inflammatory agent(s);
(c) one or more ionic polymer(s), wherein the composition has a pH in the range of 4 to 4.8.

In yet another aspect, the present invention provides a stable topically administrable otic pharmaceutical compositions comprising:

(a) one or more antibiotic(s);
(b) one or more steroidal anti-inflammatory agent(s);
(c) one or more ionic polymer(s), wherein the composition optionally comprises preservative(s), buffering agent(s), tonicity-adjusting agent(s), surfactant(s) and/or chelating agent(s).

In yet another aspect, the present invention provides a stable topically administrable otic pharmaceutical compositions comprising:

(a) one or more antibiotic(s);
(b) one or more steroidal anti-inflammatory agent(s);
(c) one or more ionic polymer(s), wherein the composition has an osmolality in the range from 270 to 330 mOsml/kg; a pH in the range of 4 to 4.8; and the composition optionally comprises preservative(s), buffering agent(s), tonicity-adjusting agent(s), surfactant(s) and/or chelating agent(s).

In yet another aspect, the present invention provides a stable topically administrable otic pharmaceutical suspension composition comprising one or more antibiotic(s) and one or more steroidal anti-inflammatory agent(s), the said pharmaceutical otic suspension further comprises one or more ionic polymer(s) wherein the composition has an osmolality in the range from 270 to 330 mOsml/kg; a pH in the range of 4 to 4.8; and the composition optionally comprises preservative(s), buffering agent(s), tonicity-adjusting agent(s), surfactant(s) and/or chelating agent(s).

In yet another aspect, the present invention provides a method of treating, preventing or reducing the risk of an otic infection in a patient in need thereof comprising administering a sterile, storage-stable topically administrable otic pharmaceutical compositions comprising one or more antibiotic(s) and one or more steroidal anti-inflammatory agent(s).

In yet another aspect, the present invention provides a method of treating, preventing or reducing the risk of an otic infection in a patient in need thereof comprising administering a sterile, storage-stable topically administrable otic pharmaceutical compositions comprising one or more antibiotic(s), one or more steroidal anti-inflammatory agent(s), one or more ionic polymer(s) and pharmaceutically acceptable excipients thereof.

In yet another aspect, the present invention provides a process of preparing a sterile, storage-stable topically administrable otic pharmaceutical composition comprising one or more antibiotic(s) and one or more steroidal anti-inflammatory agent(s).

In yet another aspect, the present invention provides a process of preparing a sterile, storage-stable topically administrable otic pharmaceutical composition comprising one or more antibiotic(s), one or more steroidal anti-inflammatory agent(s), one or more ionic polymer(s) and pharmaceutically acceptable excipients thereof.

In yet preferred aspect, the present invention provides a method of treating acute otitis media and acute otitis externa in a patient in need thereof comprising administering a sterile, storage-stable topically administrable otic pharmaceutical composition comprising one or more antibiotic(s), one or more steroidal anti-inflammatory agent(s).

In yet another preferred aspect, the present invention provides a method of treating acute otitis media and acute otitis externa in a patient in need thereof comprising administering a sterile, storage-stable topically administrable otic pharmaceutical composition comprising one or more antibiotic(s), one or more steroidal anti-inflammatory agent(s), one or more ionic polymer(s) and pharmaceutically acceptable excipients thereof.

In yet another preferred aspect, the present invention provides a sterile, storage-stable topically administrable otic pharmaceutical composition comprising Ciprofloxacin and Dexamethasone.

In yet another preferred aspect, the present invention provides a sterile, storage-stable topically administrable otic pharmaceutical composition comprising Ciprofloxacin, Dexamethasone, one or more ionic polymer(s) and pharmaceutically acceptable excipients thereof.

In yet another preferred aspect, the present invention provides a sterile, storage-stable topically administrable otic pharmaceutical composition comprising Ciprofloxacin, Dexamethasone, sodium carboxy methyl cellulose and pharmaceutically acceptable excipients thereof.

In yet another preferred aspect, the present invention provides a method of treating, preventing or reducing the risk of an otic infection in a patient in need thereof comprising administering a sterile, storage-stable topically administrable otic pharmaceutical compositions comprising Ciprofloxacin and Dexamethasone.

In yet another preferred aspect, the present invention provides a method of treating, preventing or reducing the risk of an otic infection in a patient in need thereof comprising administering a sterile, storage-stable topically administrable otic pharmaceutical compositions comprising Ciprofloxacin, Dexamethasone, one or more ionic polymer(s) and pharmaceutically acceptable excipients thereof.

In yet another preferred aspect, the present invention provides a process of preparing a sterile, storage-stable topically administrable otic pharmaceutical composition comprising Ciprofloxacin and Dexamethasone.

In yet another preferred aspect, the present invention provides a process of preparing a sterile, storage-stable topically administrable otic pharmaceutical composition comprising Ciprofloxacin, Dexamethasone, one or more ionic polymer(s) and pharmaceutically acceptable excipients thereof.

In yet another aspect, the present invention provides a stable topically administrable otic pharmaceutical compositions comprising:

### (a) 0.1-1% (wt.) Ciprofloxacin;

### (b) 0.05-0.5% (wt.) Dexamethasone;

(c) sodium carboxymethylcellulose in an amount not more than 0.5% (wt.), wherein the composition has an osmolality in the range from 270 to 330 mOsml/kg; a pH in the range of 4 to 4.8; and the composition optionally comprises preservative(s), buffering agent(s), tonicity-adjusting agent(s), surfactant(s), chelating agent(s) and/or pH-adjusting agent.

In yet another preferred aspect, the present invention provides a sterile, storage-stable topically administrable otic suspension comprising Ciprofloxacin and Dexamethasone.

In yet another preferred aspect, the present invention provides a sterile, storage-stable topically administrable otic suspension comprising Ciprofloxacin, Dexamethasone, one or more ionic polymer(s) and pharmaceutically acceptable excipients thereof.

In yet another preferred aspect, the present invention provides a sterile, storage-stable topically administrable otic suspension comprising Ciprofloxacin, Dexamethasone, sodium carboxy methyl cellulose and pharmaceutically acceptable excipients thereof.

In yet another preferred aspect, the present invention provides a process of preparing a sterile, storage-stable topically administrable otic suspension comprising Ciprofloxacin, Dexamethasone, one or more ionic polymer(s) and pharmaceutically acceptable excipients thereof.

In yet another preferred aspect, the present invention provides a method of treating acute otitis media and acute otitis externa in a patient in need thereof comprising administering a sterile, storage-stable topically administrable otic suspension comprising Ciprofloxacin, Dexamethasone, one or more ionic polymer(s) and pharmaceutically acceptable excipients thereof.

### DETAILED DESCRIPTION OF THE INVENTION

The present invention relates to the sterile, storage-stable topically administrable otic pharmaceutical compositions comprising one or more antibiotic(s) and one or more steroidal anti-inflammatory agent(s).

As used herein, the term “antibiotics” refers to drugs which are produced by micro-organism in nature and isolated from this natural source or synthesized by chemical process which can kill harmful microorganism and cure bacterial infections in human and animals.

As used herein, the term “steroidal anti-inflammatory agents” refers to the drugs that block the synthesis of new histamine release by mast cells, inactivate available histamine, inhibit mast cell degranulation, decrease capillary permeability, and inhibit phospholipase A synthesis, which is used in the production of arachidonic acid and thereby prevent inflammation.

The topical pharmaceutical compositions of the present invention comprises but not limited to suspensions, solutions, emulsions, ointments, liniments, lotions, creams and gels.

In one embodiment, the present invention provides a pharmaceutical composition comprising fixed dose combination comprising one or more antibiotic(s) and one or more steroidal anti-inflammatory agent(s).

In yet another embodiment, the present invention provides a pharmaceutical composition comprising fixed dose combination comprising one or more antibiotic(s), one or more steroidal anti-inflammatory agent(s) and one or more Ionic polymer(s).

In yet another embodiment, the present invention provides a sterile, storage-stable topically administrable otic pharmaceutical compositions comprising:

(a) one or more antibiotic(s),
(b) one or more steroidal anti-inflammatory agent(s),
(c) one or more Ionic polymer(s).

In yet another embodiment, the present invention provides a stable topically administrable otic pharmaceutical compositions comprising:

(a) one or more antibiotic(s);
(b) one or more steroidal anti-inflammatory agent(s);
(c) one or more ionic polymer(s), wherein the composition has an osmolality in the range from 270 to 330 mOsml/kg.

In yet another embodiment, the present invention provides a stable topically administrable otic pharmaceutical compositions comprising:

(a) one or more antibiotic(s);
(b) one or more steroidal anti-inflammatory agent(s);
(c) one or more ionic polymer(s), wherein the composition has a pH in the range of 4 to 4.8.

In yet another embodiment, the present invention provides a stable topically administrable otic pharmaceutical compositions comprising:

(a) one or more antibiotic(s);
(b) one or more steroidal anti-inflammatory agent(s);
(c) one or more ionic polymer(s), wherein the composition optionally comprises preservative(s), buffering agent(s), tonicity-adjusting agent(s), surfactant(s) and/or chelating agent(s).

In yet another embodiment, the present invention provides a method of treating, preventing or reducing the risk of an otic infection in a patient in need thereof comprising administering a sterile, storage-stable topically administrable otic pharmaceutical compositions comprising one or more antibiotic(s) and one or more steroidal anti-inflammatory agent(s).

In yet another embodiment, the present invention provides a method of treating, preventing or reducing the risk of an otic infection in a patient in need thereof comprising administering a sterile, storage-stable topically administrable otic pharmaceutical compositions comprising one or more antibiotic(s), one or more steroidal anti-inflammatory agent(s), one or more Ionic polymer(s) and pharmaceutically acceptable excipients thereof.

In yet another embodiment, the present invention provides a process of preparing a sterile, storage-stable topically administrable otic pharmaceutical composition comprising one or more antibiotic(s) and one or more steroidal anti-inflammatory agent(s).

In yet another embodiment, the present invention provides a process of preparing a sterile, storage-stable topically administrable otic pharmaceutical composition comprising one or more antibiotic(s), one or more steroidal anti-inflammatory agent(s), one or more ionic polymer(s) and pharmaceutically acceptable excipients thereof.

In yet another preferred embodiment, the present invention provides a method of treating acute otitis media and acute otitis externa in a patient in need thereof comprising administering a sterile, storage-stable topically administrable otic pharmaceutical compositions comprising one or more antibiotic(s), one or more steroidal anti-inflammatory agent(s).

In yet another preferred embodiment, the present invention provides a method of treating acute otitis media and acute otitis externa in a patient in need thereof comprising administering a sterile, storage-stable topically administrable otic pharmaceutical composition comprising one or more antibiotic(s), one or more steroidal anti-inflammatory agent(s), one or more ionic polymer(s) and pharmaceutically acceptable excipients thereof.

Examples of antibiotics according to the present invention include, but are not limited to, Ciprofloxacin, Moxifloxacin, Ofloxacin, Enoxacin Gatifloxacin, Norfloxacin, Perfloxacin, Amifloxacin, Pirfloxacin or their pharmaceutically acceptable derivatives thereof.

Examples of steroidal anti-inflammatory agents according to the present invention include, but are not limited to Dexamethasone, Hydrocortisone or their pharmaceutically acceptable derivatives thereof.

Dexamethasone can be present in any otically acceptable form having poor water solubility such that the resulting composition is a suspension composition. Suitable forms of Dexamethasone include Dexamethasone alcohol and Dexamethasone acetate.

In yet another preferred embodiment, the present invention provides a sterile, storage-stable topically administrable otic pharmaceutical composition comprising Ciprofloxacin and Dexamethasone.

In yet another preferred embodiment, the present invention provides a sterile, storage-stable topically administrable otic pharmaceutical composition comprising Ciprofloxacin, Dexamethasone, one or more ionic polymer(s) and pharmaceutically acceptable excipients thereof.

In yet another embodiment, the present invention provides a method of treating, preventing or reducing the risk of an otic infection in a patient in need thereof comprising administering a sterile, storage-stable topically administrable otic pharmaceutical compositions comprising Ciprofloxacin and Dexamethasone.

In yet another embodiment, the present invention provides a method of treating, preventing or reducing the risk of an otic infection in a patient in need thereof comprising administering a sterile, storage-stable topically administrable otic pharmaceutical compositions comprising Ciprofloxacin, Dexamethasone, one or more ionic polymer(s) and pharmaceutically acceptable excipients thereof.

In yet another preferred embodiment, the present invention provides a process of preparing a sterile, storage-stable topically administrable otic pharmaceutical composition comprising Ciprofloxacin and Dexamethasone.

In yet another preferred embodiment, the present invention provides a process of preparing a sterile, storage-stable topically administrable otic pharmaceutical composition comprising Ciprofloxacin, Dexamethasone, one or more ionic polymer(s) and pharmaceutically acceptable excipients thereof.

In yet another preferred embodiment, the present invention provides a method of treating acute otitis media and acute otitis externa in a patient in need thereof comprising administering a sterile, storage-stable topically administrable otic pharmaceutical composition comprising Ciprofloxacin and Dexamethasone.

In yet another preferred embodiment, the present invention provides a method of treating acute otitis media and acute otitis externa in a patient in need thereof comprising administering a sterile, storage-stable topically administrable otic pharmaceutical composition comprising Ciprofloxacin, Dexamethasone, one or more ionic polymer(s) and pharmaceutically acceptable excipients thereof.

In yet another preferred embodiment, the present invention provides a sterile, storage-stable topically administrable otic suspension comprising Ciprofloxacin and Dexamethasone.

In yet another preferred embodiment, the present invention provides a sterile, storage-stable topically administrable otic suspension comprising Ciprofloxacin, Dexamethasone, one or more ionic polymer(s) and pharmaceutically acceptable excipients thereof.

In yet another preferred embodiment, the present invention provides a method of treating, preventing or reducing the risk of an otic infection in a patient in need thereof comprising administering a sterile, storage-stable topically administrable otic suspension comprising Ciprofloxacin and Dexamethasone.

In yet another preferred embodiment, the present invention provides a method of treating, preventing or reducing the risk of an otic infection in a patient in need thereof comprising administering a sterile, storage-stable topically administrable otic suspension comprising Ciprofloxacin, Dexamethasone, one or more ionic polymer(s) and pharmaceutically acceptable excipients thereof.

In yet another preferred embodiment, the present invention provides a process of preparing a sterile, storage-stable topically administrable otic suspension comprising Ciprofloxacin and Dexamethasone.

In yet another preferred embodiment, the present invention provides a process of preparing a sterile, storage-stable topically administrable otic suspension comprising Ciprofloxacin, Dexamethasone, one or more ionic polymer(s) and pharmaceutically acceptable excipients thereof.

In yet another preferred embodiment, the present invention provides a method of treating acute otitis media and acute otitis externa in a patient in need thereof comprising administering a sterile, storage-stable topically administrable otic suspension comprising Ciprofloxacin and Dexamethasone.

In yet another preferred embodiment, the present invention provides a method of treating acute otitis media and acute otitis externa in a patient in need thereof comprising administering a sterile, storage-stable topically administrable otic suspension comprising Ciprofloxacin, Dexamethasone, one or more ionic polymer(s) and pharmaceutically acceptable excipients thereof.

The average particle size (mean volume basis) of the Dexamethasone ingredient should be less than about 20 micrometers to avoid irritation or discomfort. Dexamethasone particles can be sized using known techniques, such as ball-milling, microfluidization, high shear homogenization, high pressure homogenization and sonication.

Ciprofloxacin can be present in any otically acceptable form such that the Ciprofloxacin is in solution in the final composition.

In yet another embodiment, the antibiotics (e.g. Ciprofloxacin or its pharmaceutically acceptable salts or hydrates or combinations thereof) are present in a composition or formulation described herein in an amount of about 0.01% (wt.) to about 10% (wt.), preferably about 0.1% (wt.) to about 1% (wt.). A preferred form of Ciprofloxacin is Ciprofloxacin hydrochloride.

In yet another embodiment, the anti-inflammatory steroids (e.g. Dexamethasone or its pharmaceutically acceptable derivatives thereof) are present in a composition or formulation described herein in an amount of about 0.01% (wt.) to about 10% (wt.), preferably about 0.05% (wt.) to about 1% (wt.).

The preferred amounts of Ciprofloxacin and Dexamethasone in the composition of the present invention are 0.3 and 0.1% (wt.), respectively.

In yet another embodiment, the topical pharmaceutical compositions of present invention have a pH from 3.0 to 7.0, preferably from 4.0 to 5.5.

In yet another embodiment, the topical pharmaceutical compositions of present invention has an osmolarity from 200 to 400 milliosmoles/liter (mOsm/L), preferably from 250 to 375 mOsm/L.

A pharmaceutical composition according to the present invention may include one or more pharmaceutically acceptable buffering agents, preservatives, tonicity-adjusting agents, antioxidants, pH-adjusting agents, chelating agents, viscosity modifiers such as ionic polymers, surfactants, solubilizing agents and the like present in an amount ranging from 0.01% w/v to about 99.9% w/v.

Examples of buffering agents include, but are not limited to, phosphate buffer such as dibasic sodium phosphate heptahydrate, monobasic sodium phosphate monohydrate; and/or borate buffer such as sodium borate, boric acid; and/or citrate buffer, acetate buffer such as sodium acetate or its hydrate(s), acetic acid or combination thereof; carbonate buffer; borate-polyol complexes, and the like.

Examples of preservatives include, but are not limited to, benzalkonium chloride, benzethonium chloride, p-oxybenzoates such as methyl p-oxybenzoate or ethyl p-oxybenzoate, benzyl alcohol, phenethyl alcohol, sorbic acid or its salt, thimerosal, chlorobutanol, other quaternary amines and the like, chlorhexidine gluconate and the like.

Examples of tonicity-adjusting agents include, but are not limited to, ionic tonicity agents and non-ionic tonicity agents.

Examples of ionic tonicity agents include, but are not limited to, sodium chloride, potassium chloride, magnesium chloride or calcium chloride and the like.

Examples of non-ionic tonicity agents include, but are not limited to, mannitol, glycerine dextrose and the like.

Examples of antioxidants include, but are not limited to, ascorbic acid, malic acid, citric acid, sodium citrate, butylated hydroxyanisole, butylated hydroxytoluene, propyl gallate, sodium ascorbate, sodium metabisulfite and the like and mixtures thereof.

Examples of the alkaline agents that may be used as pH adjusting agents, include, but are not limited to, sodium hydroxide (NaOH), potassium hydroxide (KOH), tromethamine, monoethanolamine, sodium bicarbonate (NaHCO₃) and other organic and inorganic bases.

Examples of the acidic agents that may be used as pH adjusting agents include, but are not limited to, hydrochloric acid, citric acid, tartaric acid, lactic acid and other organic and inorganic acids and the like and mixtures thereof.

Examples of chelating agents include, but are not limited to, EDTA, disodium edetate, sodium citrate, condensed sodium phosphate and the like.

Examples of viscosity modifiers chosen from ionic polymers include, but are not limited to; carboxyvinyl polymer or polyacrylic acid which are commercially available under the tradename Carbopol and sodium carboxymethylcellulose which is commercially available under the tradename Cekol. Examples of sodium carboxymethylcellulose for use herein include various grades of Cekol such as, for example Cekol 700P and Cekol 150.

Examples of surfactants include, but are not limited to, cetylpyridinium chloride, tyloxapol, and various polysorbates such as Tween® which includes Tween 80 (Polysorbate 80), polyethoxylated substances and poloxamers.

Examples of solubilizing agents include, but are not limited to, cyclodextrins (CDs) such as Hydroxypropyl beta-CD, hydroxypropyl gamma-CD (Cavasol®), sulfobutyl ether4 beta-CD (Captisol®), and hydroxypropyl beta-CD (Kleptose®).

In yet another preferred embodiment, the present invention provides a sterile, storage-stable topically administrable otic pharmaceutical compositions comprising:

### (a) Ciprofloxacin Hydrochloride;

### (b) Dexamethasone;

(c) Sodium carboxymethylcellulose.

In yet another preferred embodiment, the present invention provides a sterile, storage-stable topically administrable otic suspension comprising Ciprofloxacin, Dexamethasone, sodium carboxy methyl cellulose and pharmaceutically acceptable excipients thereof.

In yet another preferred embodiment, the present invention provides a stable topically administrable otic pharmaceutical compositions comprising:

### (a) Ciprofloxacin Hydrochloride;

### (b) Dexamethasone;

(c) Sodium carboxymethylcellulose, wherein the composition has an osmolality in the range from 270 to 330 mOsml/kg.

In yet another preferred embodiment, the present invention provides a stable topically administrable otic pharmaceutical compositions comprising:

### (a) Ciprofloxacin Hydrochloride;

### (b) Dexamethasone;

(c) Sodium carboxymethylcellulose, wherein the composition has a pH in the range of 4 to 4.8.

In yet another preferred embodiment, the present invention provides a stable topically administrable otic pharmaceutical compositions comprising:

### (a) Ciprofloxacin Hydrochloride;

### (b) Dexamethasone;

(c) Sodium carboxymethylcellulose, wherein the composition optionally comprises preservative(s), buffering agent(s), tonicity-adjusting agent(s), surfactant(s) and/or chelating agent(s).

In yet another preferred embodiment, the present invention provides a stable topically administrable otic pharmaceutical composition comprising:

### (a) Ciprofloxacin Hydrochloride;

### (b) Dexamethasone;

(c) Sodium carboxymethylcellulose, one or more preservative(s), buffering agent(s), tonicity-adjusting agent(s), surfactant(s), chelating agent(s) and/or pH-adjusting agent(s); wherein the composition has an osmolality in the range from 270 to 330 mOsml/kg and a pH in the range of 4 to 4.8.

In yet another preferred embodiment, the present invention provides a stable topically administrable otic pharmaceutical suspension comprising:

### (a) Ciprofloxacin Hydrochloride;

### (b) Dexamethasone;

(c) Sodium carboxymethylcellulose, one or more preservative(s), buffering agent(s), tonicity-adjusting agent(s), surfactant(s), chelating agent(s) and/or pH-adjusting agent(s); wherein the composition has an osmolality in the range from 270 to 330 mOsml/kg and a pH in the range of 4 to 4.8.

In yet another preferred embodiment, the present invention provides a stable topically administrable otic pharmaceutical compositions comprising:

### (a) 0.1-1% (wt.) Ciprofloxacin;

### (b) 0.05-0.5% (wt.) Dexamethasone;

(c) sodium carboxymethylcellulose in an amount not more than 0.5% (wt.), one or more preservative(s), buffering agent(s), tonicity-adjusting agent(s), surfactant(s), chelating agent(s) and/or pH-adjusting agent(s); wherein the composition has an osmolality in the range from 270 to 330 mOsml/kg and a pH in the range of 4 to 4.8.

In yet another preferred embodiment, the present invention provides a stable topically administrable otic pharmaceutical suspension comprising:

### (a) 0.1-1% (wt.) Ciprofloxacin;

### (b) 0.05-0.5% (wt.) Dexamethasone;

(c) sodium carboxymethylcellulose in an amount not more than 0.5% (wt.), one or more preservative(s), buffering agent(s), tonicity-adjusting agent(s), surfactant(s), chelating agent(s) and/or pH-adjusting agent(s); wherein the composition has an osmolality in the range from 270 to 330 mOsml/kg and a pH in the range of 4 to 4.8.

In yet another preferred embodiment, the present invention provides a stable topically administrable otic pharmaceutical composition comprising:

### (a) 0.3% (wt.) Ciprofloxacin;

### (b) 0.1% Dexamethasone;

(c) sodium carboxymethylcellulose in an amount not more than 0.5% (wt.), one or more preservative(s), buffering agent(s), tonicity-adjusting agent(s), surfactant(s), chelating agent(s) and/or pH-adjusting agent(s); wherein the composition has an osmolality in the range from 270 to 330 mOsml/kg and a pH in the range of 4 to 4.8.

In yet another preferred embodiment, the present invention provides a stable topically administrable otic pharmaceutical suspension comprising:

### (a) 0.3% (wt.) Ciprofloxacin;

### (b) 0.1% Dexamethasone;

(c) sodium carboxymethylcellulose in an amount not more than 0.5% (wt.), one or more preservative(s), buffering agent(s), tonicity-adjusting agent(s), surfactant(s), chelating agent(s) and/or pH-adjusting agent(s); wherein the composition has an osmolality in the range from 270 to 330 mOsml/kg and a pH in the range of 4 to 4.8.

In yet another preferred embodiment, the present invention provides a stable topically administrable otic pharmaceutical composition comprising:
(a) 0.3% (wt.) Ciprofloxacin;
(b) 0.1% (wt.) Dexamethasone;
(c) sodium carboxymethylcellulose in an amount not more than 0.5% (wt.);
(d) Benzalkonium chloride;
(e) Boric acid;
(f) Sodium chloride;
(g) Tyloxapol;
(h) Acetic acid;
(i) Sodium acetate trihydrate;
(j) Edetate disodium;
(k) Hydrochloric acid and or Sodium Hydroxide, wherein the composition has an osmolality in the range from 270 to 330 mOsml/kg and has pH in the range of 4 to 4.8.

In yet another preferred embodiment, the present invention provides a stable topically administrable otic pharmaceutical suspension comprising:
(a) 0.3% (wt.) Ciprofloxacin;
(b) 0.1% (wt.) Dexamethasone;
(c) sodium carboxymethylcellulose in an amount not more than 0.5% (wt.);
(d) Benzalkonium chloride;
(e) Boric acid;
(f) Sodium chloride;
(g) Tyloxapol;
(h) Acetic acid;
(i) Sodium acetate trihydrate;
(j) Edetate disodium;
(k) Hydrochloric acid and/or Sodium Hydroxide, wherein the composition has an osmolality in the range from 270 to 330 mOsml/kg and has pH in the range of 4 to 4.8.

In yet another embodiment, the topically administrable otic pharmaceutical compositions of present invention is sterilized using methods such as heat sterilization methods including dry heat sterilization and autoclaving (steam sterilization), gaseous sterilization methods including filtration sterilization methods, ethylene oxide sterilization and radiation sterilization.

In yet another embodiment, the present invention provides a process of preparing a sterile, storage-stable topically administrable otic pharmaceutical composition comprising the steps of:
(a) preparing an aqueous solution of ionic polymer and autoclaving said solution;
(b) Preparing a slurry comprising steroidal anti-inflammatory agent(s) and half quantity of surfactant and water and autoclaving said slurry;
(c) Preparing a solution comprising antibiotic(s), Buffer(s), Chelating agent(s), Preservative(s), Tonicity-adjusting agent(s) and half quantity of surfactant;
(d) Adjusting the pH of the solution obtained in step (c) and making up the volume up to 65% of desired batch size followed by aseptically filtering said solution;
(e) Mixing the solution of step (a) and slurry of step (b) to the sterile solution of step (d) with stirring, making up the volume to desired batch size followed by homogenizing to obtain a final sterile suspension.

In yet another preferred embodiment, the present invention provides a process of preparing a sterile, storage-stable topically administrable otic pharmaceutical composition comprising the steps of:
(a) Preparing an aqueous solution of sodium carboxy methyl cellulose and autoclaving said solution;
(b) Preparing a slurry comprising Dexamethasone, half quantity of surfactant and water and autoclaving said slurry;
(c) Preparing a solution comprising Ciprofloxacin Hydrochloride, Buffer(s), Chelating agent(s), Preservative(s), Tonicity-adjusting agent(s) and half quantity of Surfactant;
(d) Adjusting the pH of the solution obtained in step (c) and making up the volume up to 65% of desired batch size followed by aseptically filtering said solution;
(e) Mixing the solution of step (a) and slurry of step (b) to the sterile solution of step (d) with stirring, making up the volume to desired batch size followed by homogenizing to obtain a final sterile suspension.

In yet another embodiment, the present invention provides a method of treating, preventing or reducing the risk of an otic infection in a patient in need thereof comprising administering a sterile, storage-stable topically administrable otic pharmaceutical suspension comprising:
(a) Ciprofloxacin Hydrochloride;
(b) Dexamethasone;
(c) Sodium carboxymethylcellulose, wherein the composition optionally comprises preservative(s), buffering agent(s), tonicity-adjusting agent(s), surfactant(s) and/or chelating agent(s).

It is to be understood for the purpose of invention that the sterile topical pharmaceutical compositions of invention can be stored in different types of containers and packaging which are well known in the art. Such types of containers and packaging keeps the sterile topical pharmaceutical compositions stable for adequate period of time.

In yet another embodiment, the topical pharmaceutical compositions of present invention are thermally stable at room temperature, at 25° C. with relative humidity 40% for at least 6 months.

In yet another preferred embodiment, the present invention provides a stable topically administrable otic pharmaceutical suspension comprising:

### (a) 0.3% (wt.) Ciprofloxacin;

### (b) 0.1% Dexamethasone;

(c) sodium carboxymethylcellulose in an amount not more than 0.5% (wt.), preservative(s), buffering agent(s), tonicity-adjusting agent(s), surfactant(s), chelating agent(s) and/or pH-adjusting agent; wherein the composition has an osmolality in the range from 270 to 330 mOsml/kg and a pH in the range of 4 to 4.8; wherein the composition comprises not more than 2.6% of 20-carboxy-17-desoxy related compound when stored under the condition of 25° C.±2° C./40% RH±5% RH for 6 months.

In yet another embodiment, the topical pharmaceutical compositions of present invention are found to be sterile when sterility test performed as per United States Pharmacopoeia (USP) Chapter <71>.

In yet another embodiment, the topical pharmaceutical compositions of present invention when subjected for Resuspension time in “real time” settling studies exhibits resuspendability comparable to marketed formulations like CiproDex®.

### EXAMPLES

The invention will be further illustrated by the following examples, which are intended to be illustrative but not limiting.

### Example No. 1

TABLE NO. 1
Otic Suspension containing Ciprofloxacin Hydrochloride
Eq. to Ciprofloxacin (0.3% w/v dissolved) and Dexamethasone
(0.1% suspended)

| Sr. No. | Name of Pharmaceutical ingredient | Quantity (in mg/ml) |
|---|---|---|
| 1. | Ciprofloxacin hydrochloride | 3.50 |
| 2. | Dexamethasone (Micronized) | 1.00 |
| 3. | Benzalkonium chloride | 0.10 |
| 4. | Boric acid | 6.00 |
| 5. | Sodium chloride | 5.30 |
| 6. | Sodium carboxymethylcellulose | 3.80 |
| 7. | Tyloxapol | 0.50 |
| 8. | Acetic acid | 0.40 |
| 9. | Sodium acetate trihydrate | 0.30 |
| 10. | Edetate disodium | 0.10 |
| 11. | Hydrochloric acid | q.s |
| 12. | Sodium Hydroxide | q.s |
| 13. | Water for injection | q.s to 1 mL |

### Manufacturing Process:

The formulation as shown in table 1 was prepared as follows:

1. Sodium carboxy methyl cellulose was dissolved in sufficient quantity of water for injection and sterilized by using Autoclave.
2. Accurately weighed quantity of Dexamethasone and half quantity of Tyloxapol was added in sufficient quantity of water to form slurry and sterilized by using Autoclave.
3. Accurately weighed quantities of Ciprofloxacin hydrochloride, Edetate disodium, and Sodium chloride, Sodium acetate trihydrate, Boric acid, Tyloxapol, Acetic acid, Benzalkonium chloride were successively added in sufficient quantity of water and stirred till clear a solution was obtained, desired pH was adjusted using Hydrochloric acid/Sodium hydroxide and the volume was made up to the 65% of the desired batch size. This solution was then filtered and sterilized using sterilization in place (SIP) technique.
4. The solution as obtained in step-1 and the slurry as obtained in step-2 were added the solution as obtained in step-3 and stirred and finally volume made up to the desire batch size.
5. The composition as obtained in step-4 was homogenized to obtained final sterile suspension and filed into suitable container.

### Example No. 2

TABLE NO. 2
Otic Suspension containing Ciprofloxacin Hydrochloride
Eq. to Ciprofloxacin (0.3% w/v dissolved) and Dexamethasone
(0.1% suspended)

| Sr. No. | Name of Pharmaceutical ingredient | Quantity (in mg/ml) |
|---|---|---|
| 1. | Ciprofloxacin hydrochloride | 3.50 |
| 2. | Dexamethasone (Micronized) | 1.00 |
| 3. | Benzalkonium chloride | 0.10 |
| 4. | Sodium chloride | 5.30 |
| 5. | Boric acid | 6.00 |
| 6. | Tyloxapol | 0.50 |
| 7. | Sodium carboxy methyl cellulose | 4.51 |
| | (Cekol 700P) | |
| 8. | Edetate disodium | 0.10 |
| 9. | Sodium acetate | 0.30 |
| 10. | Acetic acid | 0.40 |
| 11. | Sodium hydroxide | q.s. |
| 12. | Hydrochloric acid | q.s. |
| 13. | Water for injection | q.s. to 1 mL |

### Example No. 3

TABLE NO. 3
Otic Suspension containing Ciprofloxacin Hydrochloride
Eq. to Ciprofloxacin (0.3% w/v dissolved) and Dexamethasone
(0.1% suspended)

| Sr. No. | Name of Pharmaceutical ingredient | Quantity (in mg/ml) |
|---|---|---|
| 1. | Ciprofloxacin hydrochloride | 3.50 |
| 2. | Dexamethasone (Micronized) | 1.00 |
| 3. | Benzalkonium chloride | 0.10 |
| 4. | Sodium chloride | 5.30 |
| 5. | Boric acid | 6.00 |
| 6. | Tyloxapol | 0.50 |
| 7. | Sodium carboxy methyl cellulose | 4.75 |
| | (Cekol 150) | |
| 8. | Edetate disodium | 0.10 |
| 9. | Sodium acetate | 0.30 |
| 10. | Acetic acid | 0.40 |
| 11. | Sodium hydroxide | q.s. |
| 12. | Hydrochloric acid | q.s. |
| 13. | Water for injection | q.s. to 1 mL |

### Example No. 4

TABLE NO. 4
Otic Suspension containing Ciprofloxacin Hydrochloride
Eq. to Ciprofloxacin (0.3% w/v dissolved) and Dexamethasone
(0.1% suspended)

| Sr. No. | Name of Pharmaceutical ingredient | Quantity (in mg/ml) |
|---|---|---|
| 1. | Ciprofloxacin hydrochloride | 3.50 |
| 2. | Dexamethasone (Micronized) | 1.00 |
| 3. | Benzalkonium chloride | 0.10 |
| 4. | Sodium chloride | 5.30 |
| 5. | Boric acid | 6.00 |
| 6. | Tyloxapol | 0.50 |
| 7. | Sodium carboxy methyl cellulose | 3.80 |
| | (Cekol 700P) | |
| 8. | Edetate disodium | 0.10 |
| 9. | Sodium acetate | 0.30 |
| 10. | Acetic acid | 0.40 |
| 11. | Sodium hydroxide | q.s. |
| 12. | Hydrochloric acid | q.s. |
| 13. | Water for injection | q.s. to 1 mL |

**q.s. means quantum sufficit (a sufficient volume), i.e. to bring the solution to volume.

### Manufacturing Process:

The formulations as shown in table 2, 3 & 4 were prepared as follows:

1. Sodium carboxy methyl cellulose was dissolved in sufficient quantity of water for injection and sterilized by using Autoclave at 121° C.
2. Accurately weighed quantity of Dexamethasone and half quantity of Tyloxapol was added in sufficient quantity of water and mixed well to form slurry and sterilized by using Autoclave at 121 PC.
3. Accurately weighed quantities of Ciprofloxacin hydrochloride, Edetate disodium, and Sodium chloride, Sodium acetate trihydrate, Boric acid, Acetic acid, Benzalkonium chloride, half quantity of Tyloxapol were successively added in sufficient quantity of water and stirred till a clear solution was obtained; desired pH was adjusted using Hydrochloric acid/Sodium hydroxide and the volume was made up to the 65% of the desired batch size. This solution was then filtered and sterilized using sterilization in place (SIP) technique at 121° C.
4. The solution as obtained in step-1 and the slurry as obtained in step 2 were added to the solution as obtained in step-3 and stirred and finally volume made up to the desire batch size.
5. The composition as obtained in step-4 was homogenized to obtained final sterile suspension and filed into suitable container.

### Stability Tests:

The otic suspension of Example 4 was found stable for the period of 6 month when stored under conditions of 25° C.±2° C./40% RH±5% RH. Table no. 5 contains result of the stability studies performed on otic suspension of Example 4.

TABLE NO. 5
Otic Suspension containing Ciprofloxacin Hydrochloride Eq. to Ciprofloxacin (0.3% w/v
dissolved) and Dexamethasone (0.1% suspended) finished product analysis data-initial
and on stability

| | | | Stability Conditions | | |
|---|---|---|---|---|---|
| | | | | 25° C. ± 2° C./ | 25° C. ± 2° C./ |
| | | | | 40% | 40% |
| | | | | RH ± 5% | RH ± 5% |
| | | | | RH for | RH for 6 |
| Tests | | Specifications | Initial | 3 months | months |
| Description | | White to off | White | White | White |
| | | white | suspension | suspension | suspension |
| | | suspension | | | |
| pH | | Between 3.8 to | 4.6 | 4.2 | 4.2 |
| | | 4.8 | | | |
| Viscosity | | Between 3.0 cps | 4.6 | 4.4 | 4.5 |
| | | to 6.0 cps | | | |
| Osmolality | | Between 270 | 306 | 302 | 306 |
| | | to 330 mOsmol/kg | | | |
| Assay | Ciprofloxacin | NLT 90.0% | 102.0 | 102.6 | 102.3 |
| (By HPLC) | | and NMT | | | |
| | | 110.0% of the | | | |
| | | label claim | | | |
| | Dexamethasone | NLT 90.0% | 103.7 | 103.1 | 104.3 |
| | | and NMT | | | |
| | | 110.0% of the | | | |
| | | label claim | | | |
| Assay of Benzalkonium chloride | | NLT 60.0% | 94.2 | 99.2 | 103.0 |
| (By HPLC) | | and NMT | | | |
| | | 120.0% of the | | | |
| | | label claim | | | |
| Assay of EDTA (By HPLC) | | NLT 60.0% | 102.5 | 99.7 | 101.1 |
| | | and NMT | | | |
| | | 120.0% of the | | | |
| | | label claim | | | |
| Assay of Boric Acid (By HPLC) | | NLT 80.0% | 103.6 | 103.9 | 101.1 |
| | | and NMT | | | |
| | | 120.0% of the | | | |
| | | label claim | | | |
| Sterility | | Should be | Complies | NA | NA |
| (By direct inoculation method) | | sterile | | | |
| Content of Ciprofloxacin | | Not more than | 0.006 | 0.03 | 0.06 |
| formamide (By HPLC) | | 0.5% | | | |
| Related | Ciprofloxacin | Not more than | ND | ND | ND |
| Substances for | Ethylenediamine | 0.2% | | | |
| Ciprofloxacin | Other single | NMT 0.2% | ND | ND | ND |
| (By HPLC) | related | | | | |
| | compound | | | | |
| | Sum of all | NMT 0.8% | 0.04 | 0.09 | 0.1 |
| | related | | | | |
| | compound | | | | |
| Related | 21-dehydro-17- | NMT 1.0% | 0.1 | 0.3 | 0.4 |
| Substances for | deoxy related | | | | |
| Dexamethasone | compound | | | | |
| (By HPLC) | 20-carboxy-17- | NMT 2.6% | ND | 0.2 | 0.3 |
| | desoxy related | | | | |
| | compound | | | | |
| | Other related | NMT 0.3% | 0.04 | 0.05 | 0.06 |
| | compound | | | | |
| | Sum of all related | NMT 3.5% | 0.2 | 0.7 | 0.9 |
| | compounds | | | | |

ND: Not Detected,
NA: Not Applicable,
NLT: Not less than,
NMT: Not more than

### Resuspendability Test:

Real time settling studies were performed by allowing samples of Example 4 in measuring cylinder to undergo natural settling (under gravity) for seven days. The resuspendability of the settled material is tested by measuring the number of inversions required to fully re-suspend the sediment.

Table no. 6 contains the result of resuspendability carried out on otic suspension of Example 4.

TABLE NO. 6
Result of Resuspendability test carried out on otic
suspension of Example 4.

| | | Real-Time settling number of |
|---|---|---|
| Sr. | | inversions for complete |
| No. | Formulation | resuspension |
| 1 | Formulation prepared according to | 5 |
| | Example 4 | |

What is claimed is:

1. A stable topically administrable otic pharmaceutical suspension composition comprising one or more antibiotic(s), one or more steroidal anti-inflammatory agent(s), one or more ionic polymer(s) and optionally one or more preservative(s), buffering agent(s), tonicity-adjusting agent(s), surfactant(s) and/or chelating agent(s), wherein the composition has an osmolality in the range from 270 to 330 mOsml/kg and a pH in the range of 4 to 4.8.

2. The composition of claim 1, wherein the antibiotic(s) selected from the group consisting of Ciprofloxacin, Moxifloxacin, Ofloxacin, Enoxacin Gatifloxacin, Norfloxacin, Perfloxacin, Amifloxacin, Pirfloxacin and pharmaceutically acceptable derivatives thereof.

3. The composition of claim 1, wherein the steroidal anti-inflammatory agent(s) selected from the group consisting of Dexamethasone, Hydrocortisone and pharmaceutically acceptable derivatives thereof.

4. The composition of claim 1, wherein the ionic polymer is sodium carboxymethylcellulose.

5. A stable topically administrable otic pharmaceutical compositions comprising:
(a) Ciprofloxacin or its pharmaceutically acceptable salts or hydrates or combinations thereof;
(b) Dexamethasone or its pharmaceutically acceptable derivatives thereof;
(c) one or more ionic polymer(s),
(d) one or more preservative(s), buffering agent(s), tonicity-adjusting agent(s), surfactant(s), chelating agent(s) and/or pH-adjusting agent(s), wherein the composition has an osmolality in the range from 270 to 330 mOsml/kg and a pH in the range of 4 to 4.8

6. The composition of claim 5, wherein the ionic polymer is sodium carboxymethylcellulose.

7. A stable topically administrable otic pharmaceutical compositions comprising:
(a) 0.1-1% (wt.) Ciprofloxacin;
(b) 0.05-0.5% (wt.) Dexamethasone;
(c) Sodium carboxymethylcellulose in an amount not more than 0.5% (wt.);
(d) one or more preservative(s), buffering agent(s), tonicity-adjusting agent(s), surfactant(s), chelating agent(s) and/or pH-adjusting agent(s), wherein the composition has an osmolality in the range from 270 to 330 mOsml/kg; a pH in the range of 4 to 4.8

8. The composition of claim 7 is sterilized by using one or more methods selected from heat sterilization, filtration sterilization, gaseous sterilization or radiation sterilization.

9. The composition of claim 7, wherein the composition is prepared by the process comprising the steps of:
(a) Preparing an aqueous solution of sodium carboxy methyl cellulose and autoclaving said solution;
(b) Preparing a slurry comprising Dexamethasone, half quantity of surfactant and water and autoclaving said slurry;
(c) Preparing a solution comprising Ciprofloxacin Hydrochloride, Buffer(s), Chelating agent(s), Preservative(s), Tonicity-adjusting agent(s) and half quantity of Surfactant;
(d) Adjusting the pH of the solution obtained in step (c) and making up the volume up to 65% of desired batch size followed by aseptically filtering said solution.
(e) Mixing the solution of step (a) and slurry of step (b) to the sterile solution of step (d) with stirring, making up the volume to desired batch size followed by homogenizing to obtain a final sterile suspension.

10. A method of treating, preventing or reducing the risk of an otic infection in a patient in need thereof comprising administering the composition of claim 7 to the patient.
